(19) United States
(12) Patent Application Publication
(10) Pub. No.: US 2015/0224214 A1
(43) Pub. Date: Aug. 13, 2015

(54) SYSTEM AND METHOD FOR DISINFECTING SURFACES IN A ROOM

(71) Applicant: GOJO Industries, Inc, Akron, OH (US)

(72) Inventor: Keith Allen Pelfrey, Wadsworth, OH (US)

(73) Assignee: GOJO INDUSTRIES, INC, Akron, OH (US)

(21) Appl. No.: 14/617,582

(22) Filed: Feb. 9, 2015

Related U.S. Application Data
(60) Provisional application No. 61/938,335, filed on Feb. 11, 2014.

(51) Int. Cl. A61L 2/08

(52) U.S. Cl. CPC A61L 2/088

(57) ABSTRACT

A method of neutralizing pathogens includes covering a surface to be disinfected with a coating including one or more types of minutely configured particles having a generally high conductivity. The surface is exposed to electromagnetic waves for a duration of time up to three minutes thereby killing the pathogens.

### RELATED APPLICATIONS

This patent application claims priority to patent application Ser. No. 61/938,335, titled SYSTEM FOR DISINFECTING SURFACES IN A ROOM, filed on Feb. 11, 2014 which is incorporated herein by reference in its entirety.

### FIELD OF THE INVENTION

The current invention relates generally to systems and methods for disinfecting surfaces of wide open areas, and in particular to systems using electromagnetic wave generators to disinfect surfaces in a room.

### BACKGROUND OF THE INVENTION

People entering healthcare facilities, like for example hospitals or nursing homes, frequently acquire infections during the course of their stay. Such infections, which the patients did not originally possess prior to entering the healthcare facility, are termed HAIs (Healthcare-Associated Infections) and represent a serious risk to the health of the patient. In many instances, HAIs result in prolonged stay and additional treatment, and in other instances even loss of life. In addition to the trauma imposed on the patient and their family, HAIs add significant financial burden to the healthcare facility, which incurs the cost of treatment.

As a result, healthcare administrators are acutely aware of the need to ensure that equipment surfaces and rooms are thoroughly cleaned after every use. Because of the resilience of certain bacteria and viruses, cleaning staff use strong chemicals like chlorine bleach (sodium hypochlorite) when turning over a room. Bleach denatures proteins in micro-organisms and is therefore effective as a disinfectant. However, prolonged exposure is caustic to humans, e.g. mucous membranes, and repeated use deteriorates the surface of the materials that it is used on. Moreover, the effectiveness of cleaning with fluids, whether bleach or quaternary ammonium sanitizers, depends on the user having access to reach all surfaces. Recessed areas may be particularly hard to reach effectively. It is possible in certain circumstances that cleaning fluid doesn't directly contact all germ laden surfaces.

Additionally, the time it takes to clean a hospital room with liquid disinfectants is not cost effective and contributes to delays when admitting patients or scheduling medical procedures. Typically, hospitals allot 30 to 45 minutes for cleaning staff to turn over a room. However, hospitals are constantly seeking to reduce labor costs and frequently operate with minimal staffing. This delays the availability of rooms. In some circumstances, patients on gurneys have been placed in hallways or other areas waiting for a room to be cleaned.

What is needed is a way of quickly and effectively disinfecting the surfaces of a room without the use of manually applied cleaning solutions. The embodiments of the invention described herein obviate the aforementioned situations.

### SUMMARY OF THE INVENTION

In one embodiment of the subject invention, a method of disinfecting a room includes providing an electromagnetic wave generator capable of producing electromagnetic waves in short durations and providing a coating that includes one or more types of minutely configured (or microscopically sized), highly conductive particles that can be rapidly heated by the electromagnetic waves. The method further includes applying the coating to a surface to be disinfected and subsequently exposing the coated surface to electromagnetic energy.

In another aspect of the subject invention, the minutely configured particles comprise at least one of copper nanoparticles or aluminum nanoparticles.

In yet another aspect of the subject invention, the microscopically sized particles comprise carbon nanoparticles.

In another embodiment of the subject invention, a disinfecting system includes a magnetron capable of producing microwaves in short durations and a coating having incorporated therein one or more types of nanoparticles having a substantially high conductivity.

In one aspect of the subject invention, the coating includes a binder which incorporates the nanoparticles and adheres the nanoparticles to the surface to be disinfected.

In another aspect of the subject invention, the disinfecting system further comprises an applicator for applying the coating a surface to be disinfected.

In another embodiment of the subject invention, a method of neutralizing pathogens in a room occupied by human beings includes the steps of providing an applicator for dispensing a coating having minutely configured particles, applying a coating to exposed surfaces in a room that a human being may come into contact with, placing an electromagnetic wave generator in the room, and activating the electromagnetic wave generator thereby exposing the room to electromagnetic energy.

In one aspect of the method of the subject invention, the electromagnetic wave generator is activated for a duration of time sufficient to neutralize any associated pathogens on the coated surfaces in the room.

The method of the subject invention may also include providing a coating that is comprised of electrically conductive minutely configured particles and/or a binder that adheres the electrically conductive minutely configured particles onto a surface.

The method of the subject invention may separately include providing a coating comprised of electrically conductive minutely configured particles, a binder that adheres the electrically conductive minutely configured particles onto a surface and optionally a thickening agent for increasing the viscosity of the coating.

In one aspect of the subject invention, the coating includes minutely configured particles that have substantially rounded edges, which may be comprised of metallic or carbon nanoparticles.

In still another aspect of the subject invention, the method of the subject invention includes providing a pressurized applicator for spraying a coating having minutely configured particles.

The subject invention may further include exposing the room to electromagnetic energy for a duration of time less than three (3) minutes.

The subject invention may also include exposing the room to electromagnetic energy for a duration of time less than one (1) minute.

In still yet another aspect of the subject invention, the electromagnetic wave generator is capable of exposing surfaces in the room to electromagnetic energy in the range between 300 MHz and 300 GHz.

The method of the subject invention may further include providing a mobile electromagnetic wave generator attached to a base having wheels, wherein the electromagnetic wave generator is capable of being rolled into and out of the room.

The method of the subject invention may also include a mobile electromagnetic wave generator that includes a standard electrically conductive wall outlet plug, wherein the mobile electromagnetic wave generator draws power to expose the room to electromagnetic energy from an associated standard electrically conductive wall outlet in the room.

In yet another aspect of the subject invention, a method of neutralizing pathogens in a room occupied by human beings includes the steps of providing a surface temperature sensor capable of measuring the temperature of a surface in the room covered with the coating, and monitoring the temperature of the surface in the room.

The method of the subject invention may further include the step of exposing the room to electromagnetic energy until the monitored temperature of the coated surface in the room exceeds the temperature necessary to kill Clostridium difficile bacteria.

The method of the subject invention may also include the step of providing a controller operatively communicated to the surface temperature sensor and operatively connected to deactivate the electromagnetic wave generator, communicating data from the surface temperature sensor to the controller and automatically deactivating the electromagnetic wave generator when data from the surface temperature sensor representing a predetermined threshold surface temperature has been communicated to the controller.

### BRIEF DESCRIPTION OF THE DRAWINGS

FIG. 1 depicts a wide area disinfecting system according to the embodiments of the subject invention.

FIG. 2 depicts a user applying a coating to surfaces to be disinfected by the wide area disinfecting system according to the embodiments of the subject invention.

FIG. 3 is a flow chart depicting the method of one embodiment of the subject invention.

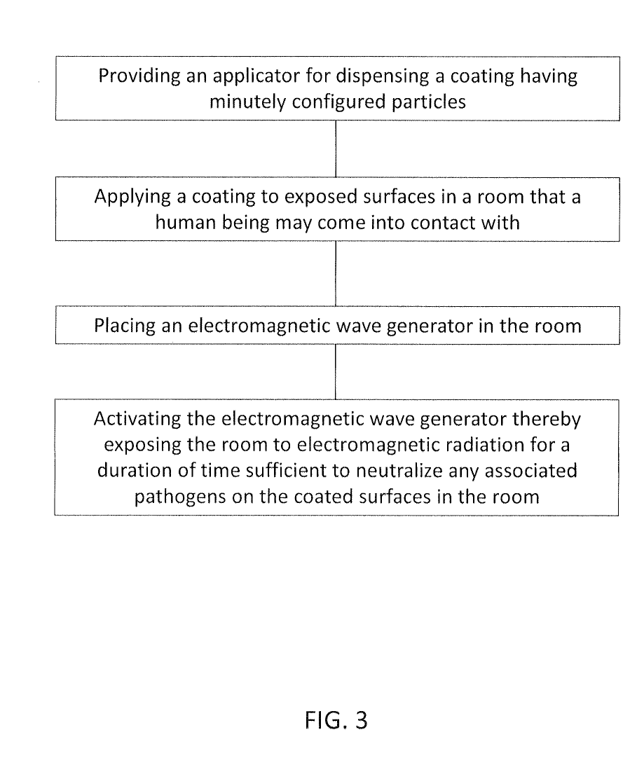

FIG. 4 is a flow chart depicting the method of another embodiment of the subject invention.

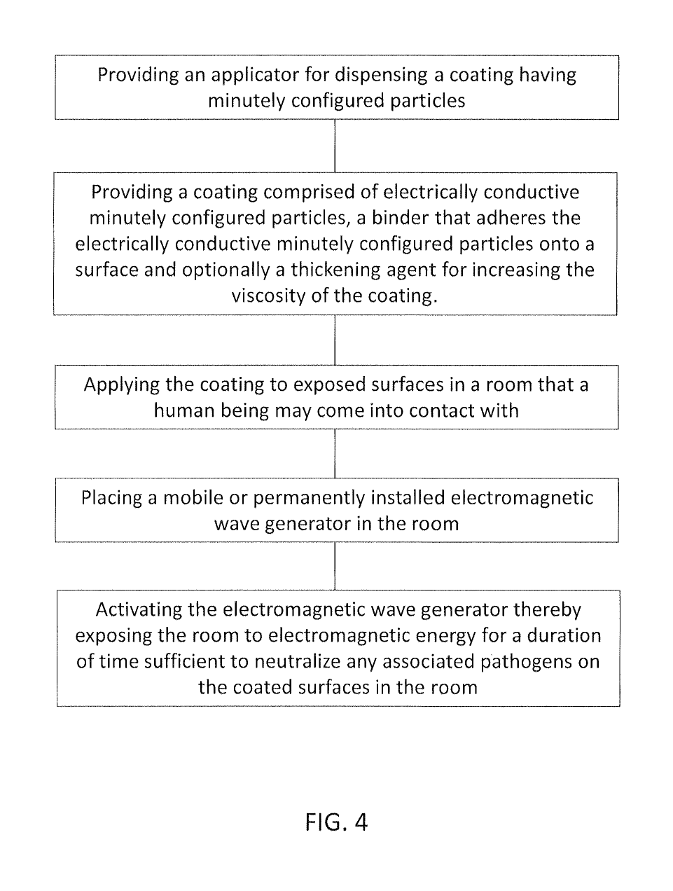

### DETAILED DESCRIPTION

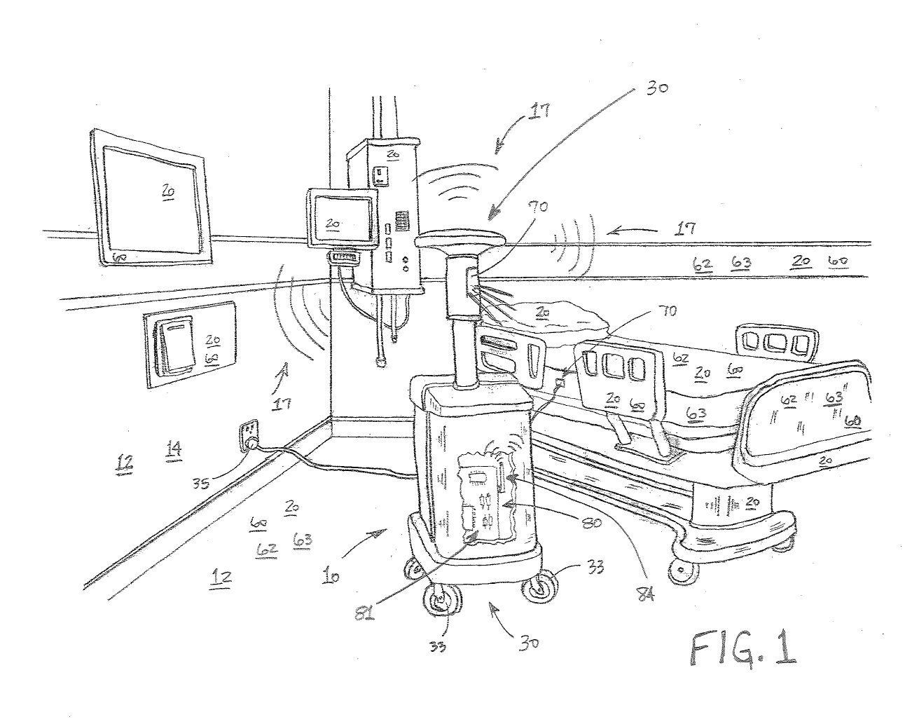

A wide area disinfecting system or whole room disinfecting system, designated generally at 10, is depicted in FIG. 1 according to the embodiments of the subject invention. The disinfecting system 10 emits energy waves that impinge on the surface of walls and other objects in a room for disinfecting purposes. In one exemplary instance, the disinfecting system 10 is a mobile or portable device that can be easily moved between rooms 12 or areas sectioned off by walls 14 or dividers, as may be found in a hospital or healthcare facility. In this way the disinfecting system 10 can be used to disinfect multiple rooms in the healthcare facility. Other embodiments are contemplated where the disinfecting system 10 may be permanently installed in the room 12 where surfaces are routinely disinfected. While specific embodiments are described in the context of healthcare facilities, persons of skill in the art will understand the application to other industries like, but not limited to, food service.

The disinfecting system 10 may function to disinfect surfaces without direct manual application of a cleaning or disinfecting solution, like chlorine Bleach or Quaternary Ammonium sanitizer. In particular, the disinfecting system 10 emit waves of energy, i.e. electromagnetic radiation or electromagnetic energy, designated generally at 17, that directly contact the surfaces 20 of various objects in a room. One such example includes a hospital room bed. The energy waves 17 are absorbed by the surfaces 20 resulting in a temperature increase. The type of energy waves emitted and duration thereof are controlled to raise the temperature of the surfaces 20 high enough to kill residing pathogens. Notably, the temperature increase is maintained just long enough, and high enough, to ensure that all residing pathogens are neutralized. In this way, any negative effects of heating the substrate are minimized. In one embodiment, surfaces 20 to be disinfected may be coated with a layer of particles that assist with energy absorption and thus the rapid increase of temperature, to be discussed further below.

Still referencing FIG. 1, the disinfecting system 10 employs an electromagnetic wave generator 30. The generator 30 functions to emit electromagnetic energy within the range of frequencies extending from the Radio frequency band (measured in KHz) through the Infrared spectrum (measured in THz, i.e. Terahertz). In one particular embodiment, the electromagnetic wave generator may emit electromagnetic waves in the range between 300 MHz and 300 GHz or in the Microwave band of frequencies. However, frequencies above and below this range may be used as is needed to effectively neutralize pathogens.

The electromagnetic wave generator 30 may use a magnetron, not shown in the figures, to produce electromagnetic waves of the desired frequency. One type of magnetron converts high power DC input voltage into RF energy using a series of cavity resonators. A cathode and one or more anodes are connected to the DC voltage source. The anode is positioned at a center of the cavities, which may be oriented in a generally cylindrical fashion. A magnetic field is induced or applied via permanent magnets around the cavities surrounding the anode. Electrons that leave the cathode are accelerated toward the anode as a result of the electric field established by the voltage source. The motion of the electrons are constrained and grouped together as they flow by the combined effect of the radial electrostatic field and an axial magnetic field. An antennae extends from one of the cavities, which in certain applications may be connected to one or more wave guides that channel the electromagnetic wave in a desired manner. It is noted that the frequency of an emitted electromagnetic wave or microwave is affected by the physical dimension of the cavity. Other configurations of electromagnetic wave generators may be used including, for example, Klystron amplifiers. However, it is to be construed that any means for generating the electromagnetic waves and more specifically microwave energy may be chosen as is appropriate for use with the embodiments of the subject invention.

The electromagnetic wave generator 30 may provide output waves that are configured to impinge on surfaces 20 over a wide area. In one exemplary manner, the electromagnetic wave output emanates radially outward from a single source and is designed to contact all of the surfaces in a room 12. The room 12 may be a hospital patient room, a medical operating room or other enclosed region. Persons of skill in the art will understand that the size of the room being disinfected will vary and as such the amount of power produced by the electromagnetic wave generator 30 may be adjusted or varied depending on the size of the room.

It is contemplated in an alternate embodiment that multiple electromagnetic wave generators 30 may be used in the process of disinfecting a room 12. For larger rooms and/or rooms densely populated with large quantities of equipment, multiple electromagnetic wave generators 30, i.e. generator emitters, may be included and controlled to operate simultaneously. The position of the multiple electromagnetic wave generators 30 may also be adjusted to ensure that all surface areas in the room 12 are sufficiently exposed to kill any pathogens present.

In one particular embodiment, the electromagnetic wave generator 30 may be portable, as mentioned above. By portable it is meant that the device may be equipped with wheels 33, handles or other means of transporting the electromagnetic wave generator 30 from room to room. The electromagnetic wave generator 30 may further comprise an electrical connector 35, i.e. electrical connector wall outlet plug 35, and power cord designed to draw power from a standard electrical wall outlet. However, other embodiments are contemplated that incorporate an onboard power supply, not shown, capable of storing energy for providing operating power to the electromagnetic wave generator 30 without being connected to an external source of electricity.

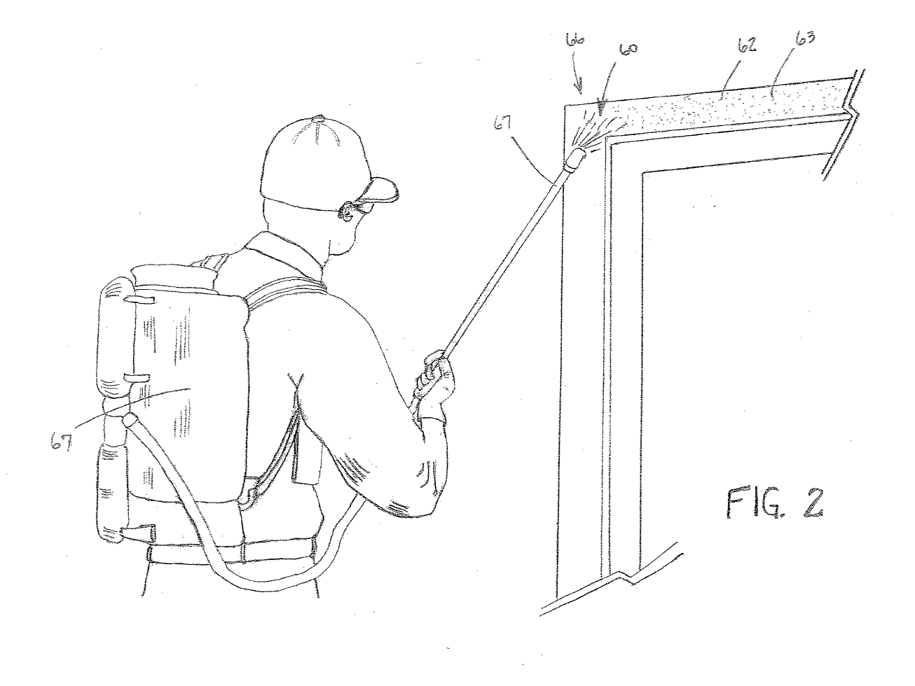

With reference to FIGS. 1 and 2, the disinfecting system 10 and methods thereof may further utilize a coating 60 that is applied to the surfaces 20 in a room 12 or area to be disinfected. The coating 60 may include one or more types of minutely configured particles 62. The particles 62 may be microscopic in size, also referred to as nanoparticles 63. In one embodiment, the nanoparticles 63 are substantially round, which is to say that the particles are free of sharp edges, which may adversely affect the absorption of the electromagnetic waves.

The coating 60 may further comprise a binder or binding agent 66. The binder 66 functions to hold the particles 62 in place after being applied. In one embodiment, the binder 66 may comprise adhesives that dry or cure forming a layer that covers the surfaces 20 to be disinfected. The coating 60 may also include one or more thickening agents that hold the particles 62 in place. The thickening agent may be used without or in conjunction with the binder 66.

Particles 62 may be constructed from materials that readily absorb electromagnetic energy and thus heat up quickly. In one embodiment, the particles 62 or nanoparticles 63 may be metallic or have similar properties of conductivity. Examples include but are not limited to: copper, silver, and aluminum. Skilled artisans will readily see the application to other types of metals. Alternatively, other types of substances may similarly be used. Carbon particles, for example, also readily absorb energy and may be sized in a similar manner. Still, any type of microscopically sized particle may be used that heats up quickly when exposed to electromagnetic energy.

The coating 60 may be applied by spraying a solution of one or more binder components in which the nanoparticles 63 are incorporated. The solution, or aerosol, may be pressurized and dispensed from an applicator. In one embodiment, the aerosol may be packaged in a disposable spray can having a spray nozzle, not shown. Alternatively, the coating may be dispensed from a refillable spray applicator 67. In this way, the binder and nanoparticles may be mixed in any desired proportion as may be necessary for a particular application. It is noted that coating 60 is applied to surfaces 20 that are regularly cleaned when a room is turned over.

Referencing all of the figures, the procedure for disinfecting a room 12 begins with manually applying the coating 60 by an end user, which may be maintenance personnel or other healthcare worker. In one embodiment, the coating 60 may be pressurized and sprayed onto the surfaces in the room 12. Alternatively, the coating 60 may be dispensed onto an applicator, which may be a cloth or sponge, and then manually applied to the surfaces in the room 12. In the instance where a binder 66 is used, the coating 60 is given time to dry or cure. In regular use, the coating 60 is applied only periodically between exposures to electromagnetic waves 17. In any case, after the nanoparticles 63 have been applied to the surface being disinfected, the electromagnetic wave generator 30 is moved into positioned and activated for a duration of time sufficient to kill pathogens residing on the surfaces 20. In one embodiment the length of time that the electromagnetic wave generator 30 is activated extends up to approximately three (3) minutes. In another embodiment, the electromagnetic wave generator 30 is activated for approximately one (1) minute. However, activation times between ten (10) and thirty (30) seconds may be used to killed pathogens residing on the surfaces covered by coating 60.

A surface temperature sensor 70 may be employed to measure the temperature of the surface being disinfected. In one particular embodiment, the surface temperature sensor 70 may be directed or placed at one or more specific regions where the coating 60 has been applied. Examples of surface temperature sensors 70 may include: thermocouples, thermal imaging, infrared, lasers and the like. Still, other types of surface temperature sensors may be employed as chosen with sound judgment. When the electromagnetic wave generator 30 has been activated, the surface temperature sensors 70 measure the changing temperature of the one or more specific regions where the coating has been applied. Feedback from the surface temperature sensors 70 may be displayed for the user operating the electromagnetic wave generator 30 to observe. When a surface temperature sufficiently high enough to kill pathogens residing on the surface has been reached, the user may be signaled to deactivate the electromagnetic wave generator 30. It is to be construed that other factors other than the magnitude of the surface temperature may be taken into account when signaling the user to deactivate the electromagnetic wave generator 30. One such embodiment takes into account the magnitude, or threshold, of surface temperature and the duration of time that the surface being disinfected is held at that temperature.

In other embodiments, signals or data from the surface temperature sensor 70 may be communicated to a controller 80, which controls the operation of the electromagnetic wave generator 30. The controller 80 may employ electronic circuitry 81 including one or more logic processors, memory circuits and other supporting electronic circuitry. Accordingly, the controller 80 may be programmable and may incorporate circuitry for communicating data via a hardwire connection and/or wireless communication means 84. When the electromagnetic wave generator 30 has been activated, the controller 80 may read data from the surface temperature sensor 70 and may automatically deactivate the electromagnetic wave generator 30 when an appropriate temperature and/or time duration has been reached. In this manner, the disinfecting system 10 functions to automatically deactivate when the pathogens residing on the surface being disinfected have been neutralized.

Having illustrated and described the principles of the dispensing system in one or more embodiments, it should be readily apparent to those skilled in the art that the invention can be modified in arrangement and detail without departing from such principles.

It is claimed:

1. A method of neutralizing pathogens in a room occupied by human beings, comprising the steps of:
providing an applicator for dispensing a coating having minutely configured particles;
applying a coating to exposed surfaces in a room that a human being may come into contact with;
placing an electromagnetic wave generator in the room; and,
activating the electromagnetic wave generator thereby exposing the room to electromagnetic energy for a duration of time sufficient to neutralize any associated pathogens on the coated surfaces in the room.

2. The method as defined in claim 1, further comprising the step of:
providing a coating comprised of electrically conductive minutely configured particles and a binder that adheres the electrically conductive minutely configured particles onto a surface.

3. The method as defined in claim 1, where in the step of providing a coating comprised of electrically conductive minutely configured particles and a binder, comprises the step of:
providing a coating comprised of electrically conductive minutely configured particles, a binder that adheres the electrically conductive minutely configured particles onto a surface and optionally a thickening agent for increasing the viscosity of the coating.

4. The method as defined in claim 1, wherein the minutely configured particles comprise minutely configured particles having substantially rounded edges.

5. The method as defined in claim 4, wherein the minutely configured particles are comprised of metallic nanoparticles.

6. The method as defined in claim 4, wherein the minutely configured particles are comprised of carbon nanoparticles.

7. The method as defined in claim 1, where in the step of providing an applicator for dispensing a coating having minutely configured particles, comprises the step of:
providing a pressurized applicator for spraying a coating having minutely configured particles.

8. The method as defined in claim 1, where in the step of exposing the room to electromagnetic energy for a duration of time sufficient to neutralize any associated pathogens on the coated surfaces in the room, comprises the step of:
exposing the room to electromagnetic energy for a duration of time less than 3 minutes.

9. The method as defined in claim 1, where in the step of exposing the room to electromagnetic energy for a duration of time sufficient to neutralize any associated pathogens on the coated surfaces in the room, comprises the step of:
exposing the room to electromagnetic energy for a duration of time less than 1 minute.

10. The method as defined in claim 1, wherein the electromagnetic wave generator is capable of exposing surfaces in the room to electromagnetic energy in the range between 300 MHz and 300 GHz.

11. The method as defined in claim 1, further comprising the step of:
providing a mobile electromagnetic wave generator attached to a base having wheels, wherein the electromagnetic wave generator is capable of being rolled into and out of the room.

12. The method as defined in claim 11, wherein the mobile electromagnetic wave generator includes a standard electrically conductive wall outlet plug; and,
where in the mobile electromagnetic wave generator draws power to expose the room to electromagnetic energy from an associated standard electrically conductive wall outlet.

13. The method as defined in claim 1, further comprising the step of:
providing a surface temperature sensor capable of measuring the temperature of a surface in the room covered with the coating; and,
monitoring the temperature of the surface in the room.

14. The method as defined in claim 13, where in the step of:
exposing the room to electromagnetic energy for a duration of time sufficient to neutralize any associated pathogens on the coated surfaces in the room, comprises the step of:
exposing the room to electromagnetic energy until the monitored temperature of the surface in the room exceeds the temperature necessary to kill Clostridium difficile bacteria.

15. The method as defined in claim 13, further comprising the step of:
providing a controller operatively communicated to the surface temperature sensor and operatively connected to deactivate the electromagnetic wave generator;
communicating data from the surface temperature sensor to the controller; and,
automatically deactivating the electromagnetic wave generator when data from the surface temperature sensor equals a predetermined threshold surface temperature.
